(19) United States
(12) Patent Application Publication
(10) Pub. No.: US 2021/0268049 A1
(43) Pub. Date: Sep. 2, 2021

(54) METHODS AND COMPOSITIONS FOR TREATMENT OF SOLID CANCERS AND MICROBIAL INFECTION

(71) Applicant: IMMVIRA CO., LIMITED, Shenzhen (CN)

(72) Inventors: Grace ZHOU, Shenzhen (CN); Xiaoqing CHEN, Shenzhen (CN)

(21) Appl. No.: 17/268,908

(22) PCT Filed: Aug. 16, 2018
(86) PCT No.: PCT/CN2018/096152

§ 371 Date: Feb. 16, 2021

(51) Int. Cl. A61K 35/763; C12N 7/00; A61P 35/00

(52) U.S. Cl. CPC A61K 35/763; A61P 35/00; C12N 7/00

(57) ABSTRACT

Provided is a recombinant virus comprising a fragment of exogenous polynucleotides encoding suppressor of cytokine signaling 4 (SOCS4) or a functional fragment thereof, wherein the recombinant vims expresses SOCS4 or the functional fragment once replication in a cell. Also provided are use of a recombinant oncolytic virus for preparation of therapeutic drugs of cancer and side effects of virus infection.

### FIELD OF THE INVENTION

The present invention relates to methods and compositions for treatment of microbial infection and solid cancers, and in particular, to recombinant viruses expressing SOCS4 and methods for use thereof.

### BACKGROUND

HSV-1 infected a variety of mucosal tissues, including respiratory tract and it had been reported that HSV-1 inducted pneumonia was due to the inflammatory response rather than direct cytopathic effects of the virus itself. This uncontrolled inflammatory response is the consequence of an excessive release of pro-inflammatory cytokines, which was called “cytokine storm” that was first used to describe influenza-induced cytokine over production in short time, which links to uncontrolled pro-inflammatory responses and significant immunopathology and severe disease outcome. Unfortunately, the understanding of molecules involved in cytokine storm, contribution of cytokines to pathogenesis, and therapeutic strategies to prevent or alleviate the symptoms are still insufficient.

Oncolytic viruses (OVs) which are genetically engineered to selectively replicate in and kill cancer cells, represent a new method of anti-tumor therapy. This approach is particularly attractive because of its mechanism-based selectivity, its potential for mediating tumor cell death, and its possibility to express additional therapeutic trans-genes at tumor site. Considering the fact that oncolytic viruses are design for intratumoral injection, this unique cancer therapeutics is often coupled with the anti-tumor immunity (immunovirotherapy) of host. Herpes simplex virus type 1 (HSV-1 ) based oncolytic HSV (oHSV), talimogene laherparepvec (T-VEC, Imlygic) was the FDA first approved OV. Like other OV candidates, the host response against oHSV is complex, multifaceted, and modulated by both host immunity and the tumor microenvironment, moreover, the various immune and inflammatory responses could be both beneficial and detrimental, and the induction of cytokine storm by OV delivery to particular organs such as lung is one of the increasingly recognized impediments. The development of cytokine storm with attendant pulmonary damage has been subsequently reported in various viral, bacterial, or fungal infections. Consistent observation informed that the concept of cytokine storm was much more complicated, but current understanding of the mechanism that promotes cytokine storm remains limited, and countermeasures to control the balance between appropriate cytokine release and cytokine overproduction remains relatively unexplored.

### SUMMARY

A first aspect of the invention is related to a recombinant virus comprising a fragment of exogenous polynucleotides encoding suppressor of cytokine signaling 4 (SOCS4) or a functional fragment thereof, wherein the recombinant virus expresses SOCS4 or the functional fragment once replication in a cell.

In some embodiments, the recombinant virus is a recombinant oncolytic virus carrying a fragment of exogenous polynucleotides encoding suppressor of cytokine signaling 4 (SOCS4) or a functional fragment thereof, and the cell where the virus replicates is a tumor cell. In some embodiments, the recombinant virus is a recombinant viral vector carrying a fragment of exogenous polynucleotides encoding suppressor of cytokine signaling 4 (SOCS4) or a functional fragment thereof, and the cell where the virus replicates is a normal cell.

Another aspect of the invention is related to methods for treating cancer or for reducing or eliminating side effects of oncolytic virus therapy in a subject comprising administering to the subject a therapeutically effective amount of a recombinant oncolytic virus, wherein the recombinant oncolytic virus comprises a fragment of exogenous polynucleotides encoding suppressor of cytokine signaling 4 (SOCS4) or a functional fragment thereof and expresses SOCS4 or the functional fragment once replication in a cancer cell.

Further aspects of the invention are related to methods for reducing or eliminating side effects of treatment of microbial infection in a subject comprising administering to the subject a therapeutically effective amount of a recombinant viral vector comprising a fragment of exogenous polynucleotides encoding suppressor of cytokine signaling 4 (SOCS4) or a functional fragment thereof, wherein the recombinant virus expresses SOCS4 or the functional fragment once replication in a normal cell.

In the present invention, we have reconstructed an HSV strain with SOCS4 protein insert (HSV-SOCS4) to investigate its effect of controlling induction of cytokine storm. The invention investigated several representative cytokines that play key roles, including MCP-1, IL-1β, TNF-α, IL-6, and IFN-γ, and found that their concentration of HSV-SOCS4 infected mice were much lower than that of HSV-1(F) infected mice, and HSV-SOCS4 mice showed slight lung damage, less weight loss and 100% survival rate. The invention provides a promising solution of governing the cytokines storm induced by oncolytic virus therapy.

Other aspects of the invention will be readily known from the detailed description of the invention set forth below.

### BRIEF DESCRIPTION OF DRAWINGS

FIG. 1: Sketch of HSV-SOCS4 reconstruction. (A) Confirmed SOCS4 gene was ligated into pNEWUL backbone site at Cla I/Acc I and Not I. (B) Sequence between BgIII and Pacl at pNEWUL backbone (including UL3, UL4 and SOCS4) was cleaved from pNEWUL and cloned into plasmid Pko5.1 at the same site.

FIG. 2: PCR confirmation of HSV-SOCS4 reconstruction. (A) Band1 showed the PCR product of SOCS4 (1397 bp) from pReveiver-M02. (B) DNA was extracted from reconstructed virus to perform PCR for the final confirmation, there bands between 1000 bp and 2000 bp were shown: UL4(1492 bp), UL3(1319 bp) and SOCS4(1397 bp).

FIG. 3: Cytokine production in BALF from mice infected with PBS, HSV-1-F or HSV-SOCS4. BALF from mouse (n=6) was collected on day 1, 3 and 7 after infection to perform ELISA assay. (A) Significant higher concentration of MCP-1 was detected from HSV-1-F mice than that from HSV-SOCS4 mice on all three-day times. MCP-1 production of HSV-1-F mice decreased on day 7 but only negligible difference was shown among HSV-SOCS4 mice. (B) Compared with HSV-SOCS4 mice, elevated level of IL-1β was observed of HSV-1-F mice on day1 and day3, but not on day 7. IL-1β production of HSV-1-F mice showed an uptrend-downtrend curve. (C) TNF-α level difference between HSV-1-F mice and HSV-SOCS4 mice was obvious and the highest TNF-α level tested of HSV-1-F mice was on day 1, then it declined. (D) Evident lower level of IL-6 was tested from HSV-SOCS4 mice than that from HSV-1-F mice on day 1, 3 and 7, and production was increased on day 7 for both groups. (E) IFN-γ production of HSV-1-F mice was much higher than from HSV-SOCS4 mice, and it increased on day3 and stayed at the high level on day 7. Mock mice served as negative control (NC) did not induce appreciable amount of cytokines. * indicates p value<0.05.

FIG. 4: Cytokine production in serum from mice infected with PBS, HSV-1-F or HSV-SOCS4. Serum from each mouse was collected on day 1, 3 and 7 after infection to perform ELISA assay. (A) Significant higher concentration of MCP-1 was detected from HSV-1-F mice than that from HSV-SOCS4 mice on day 1, and its production of HSV-1-F mice successively decreased but it decreased only on day 7 of HSV-SOCS4 mice. (B) IL-1β values of HSV-1-F and HSV-SOCS4 mice were similar on day1 and day3 but HSV-1-F mice showed a strong upregulation on day 7, therefore, great difference was found between two group mice. (C) TNF-α level difference between HSV-1-F mice and HSV-SOCS4 mice was obvious and the highest TNF-α level of HSV-1-F mice was showed on day7. (D) Evident lower level of IL-6 was tested from HSV-SOCS4 mice than that from HSV-1-F mice on all three-day times. Continuously increased IL-6 production was detected of HSV-1-F mice, but the increased lever showed only on day 7 of HSV-SOCS4 mice. (E) Much more IFN-γ production was tested from HSV-1-F mice, and it increased on day7 for both groups. Mock mice served as negative control (NC) did not induce appreciable amounts of cytokines. * indicates p value<0.05.

FIG. 5: Flow cytometric analysis of BALF cells from mice infected with HSV-1-F or HSV-SOCS4. BALF cells from mice were collected on day 1 and 7 after infection, stained with CD11b and run for flow cytometric analysis. (A) One representative result from each group of mice on day 1 and day 7 were showed. Number of CD11b+ cells was marked. (B) Results of mice (n=6) was shown. Predominated CD11b+ cells were detected from HSV-1-F mice and greater quantity cells were stained positive on day 1 than that on day 7 in both groups. * indicates p value<0.05.

FIG. 6: Flow cytometric analysis of spleen cells from mice infected with HSV-1-F or HSV-SOCS4. Spleen cells from mice were collected on day 1 and 7 after infection, stained with CD62L, CD8a or CD4 and run for flow cytometric analysis. (A) A representative CD8+ and CD62L+cell result from each group of mice on day 1 and day 7 were showed. Double positive cell number was indicted. (B) Results of mice (n=6) was shown. Double positive cells increased greatly from day1 to day 7 and quantity difference of double positive cells between HSV-1-F mice and HSV-SOCS4 mice was palpable on day 7. (C) One typical CD4+ and CD62L+ cell result from each group of mice on day 1 and day 7 were presented. Number of double positive cells was recorded. (D) Results of mice (n=6) was shown. Notably elevated CD4+ and CD62L+ cells were detected on day 7 and distinction of positive cell number between HSV-1-F and HSV-SOCS4 mice was obvious. * indicates p value<0.05.

FIG. 7: Viral titration and pathological analysis of infected mice lungs. After been removed from infected mice, lungs of mice with no BALF extracted was minced and supernatant was collected for viral titration analysis on monolayer Vero cells. (A) The maximum virus titre was observed on day 1, it declined thereafter, and no virus was detected on day 7. Virus load displayed obvious difference between HSV-1-F mice and HSV-SOCS4 mice on day 3. (B) Mice lungs without BALF taken were sent for pathological analysis and a representative 200×photograph was shown. On day 1, lung of HSV-SOCS4 mice barely showed pathological changes; but obvious cells infiltration with mild-to-moderate dilatation and hyperemia of local capillary was displayed of HSV-1-F mice lung. On day 7, infiltration of some immune cells and slight dilatation and hyperemia of capillary were observed of HSV-SOCS4 infected mice lung but architecture of lung alveolar wall was undisrupted; Thickened and disrupted alveolar wall with severe surrounding hyperemia was appeared of HSV-1-F mice lung and congested with immune cells. * indicates p value<0.05.

FIG. 8: Body weight and mortality of mice after infected with PBS, HSV-1-F or HSV-SOCS4. After infected via intranasal route, mice were monitored twice daily for a period of 12 days. (A) The average body weight (g) of all living mice was showed. HSV-1-F infected mice started to lose their body weight gradually on day 2 and the loss became sharply on day 7 and the final living mouse lost 50% body weight on day10. HSV-SOCS4 group mice lost weight slightly and generally kept 80% of weight on day12. (B) Survival rate of HSV-1-F mice stared to drop on day 7, then mice died rapidly, and no mouse survived on day 11. The survival rate of HSV-SOCS4 mice maintained at 100%. PBS mock mice showed no weight loss and no death. * indicates p value<0.05.

### DETAILED DESCRIPTION OF THE INVENTION

Definitions

It is to be noted that the term “a” or “an” entity refers to one or more of that entity; for example, “a cancer cell” is understood to represent one or more cancer cells. As such, the terms “a” (or “an”), “one or more,” and “at least one” can be used interchangeably herein.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on. The subject herein is preferably a human.

As used herein, phrases such as “to a patient in need of treatment” or “a subject in need of treatment” includes subjects, such as mammalian subjects, that would benefit from administration of a recombinant virus or a composition of the present disclosure used, e.g., for detection, for a diagnostic procedure and/or for treatment.

The term “therapeutically effective amount” or “pharmaceutically effective amount” as used in this specification refers to an amount of each active ingredient that can exert clinically significant effects. The pharmaceutically effective amount of the recombinant virus for a single dose may be prescribed in a variety of ways, depending on factors such as formulation methods, administration manners, age of patients, body weight, gender, pathologic conditions, diets, administration time, administration interval, administration route, excretion speed, and reaction sensitivity. For example, the pharmaceutically effective amount of the recombinant virus for a single dose may be in ranges of 0.001 to 100 mg/kg, or 0.02 to 10 mg/kg, but not limited thereto. The pharmaceutically effective amount for the single dose may be formulated into a single formulation in a unit dosage form or formulated in suitably divided dosage forms, or it may be manufactured to be contained in a multiple dosage container.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA) or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate-phosphodiester oligomer.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g, one, two, three, four, five, six, seven, eight, nine, ten, fifteen, twenty or more individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

The term “recombinant virus” or “reconstructed virus” as used in this specification refers to a virus that has been genetically altered, e.g., by the addition or insertion of a heterologous nucleic acid construct into and/or by the deletion of an endogenous polynucleotides from the genome of the virus. As used in the present application, the term refers to a recombinant oncolytic virus or a recombinant viral vector, both carrying a fragment of heterologous nucleic acid sequence encoding SOCS4 protein or a functional fragment thereof.

Oncolytic Virus

Numerous oncolytic viruses are known in the art and are described, any of which is envisioned for use in the invention. By way of example, appropriate oncolytic viruses include type 1 herpes simplex viruses, type 2 herpes simplex viruses, vesicular stomatitis viruses, oncolytic adenovirus, Newcastle disease viruses, vaccinia viruses, and mutant strains of these viruses. In one embodiment, the oncolytic virus is replication-selective or replication-competent. In one embodiment, the oncolytic virus is replication-incompetent.

The oncolytic viruses useful in the present methods and compositions are, in some embodiments, replication-selective. It is understood that an oncolytic virus may be made replication-selective if replication of the virus is placed under the control of a regulator of gene expression such as, for example, the enhancer/promoter region derived from the 5′-flank of the albumin gene. By way of example, the main transcriptional unit of an HSV may be placed under transcriptional control of the tumor growth factor-beta (TGF-β) promoter by operably linking HSV genes to the TGF-β promoter. It is known that certain tumor cells overexpress TGF-β, relative to non-tumor cells of the same type. Thus, an oncolytic virus wherein replication is subject to transcriptional control of the TGF-β promoter is replication-selective, in that it is more capable of replicating in the certain tumor cells than in non-tumor cells of the same type. Similar replication-selective oncolytic viruses may be made using any regulator of gene expression which is known to selectively cause overexpression in an affected cell. The replication-selective oncolytic virus may, for example, be an HSV-1 mutant in which a gene encoding ICP34.5 is mutated or deleted.

An oncolytic virus in accordance with the present invention can further comprise other modifications in its genome. For example, it can comprise additional DNA inserted into the U_{L}44 gene. This insertion can produce functional inactivation of the U_{L}44 gene and the resulting lytic phenotype, or it may be inserted into an already inactivated gene or substituted for a deleted gene.

The oncolytic virus may also have incorporated therein one or more promoters that impart to the virus an enhanced level of tumor cell specificity. In this way, the oncolytic virus may be targeted to specific tumor types using tumor cell-specific promoters. The term “tumor cell-specific promoter” or “tumor cell-specific transcriptional regulatory sequence” or “tumor-specific promoter” or “tumor-specific transcriptional regulatory sequence” indicates a transcriptional regulatory sequence, promoter and/or enhancer that is present at a higher level in the target tumor cell than in a normal cell. For example, the oncolytic virus for use in the invention may be under the control of an exogenously added regulator such as tetracycline.

In one embodiment, the oncolytic virus (e.g, oHSV) of the invention is engineered to place at least one viral protein necessary for viral replication under the control of a tumor-specific promoter. Or, alternatively a gene (a viral gene or exogenous gene) that encodes a cytotoxic agent can be put under the control of a tumor-specific promoter. By cytotoxic agent as used here is meant any protein that causes cell death. For example, such would include ricin toxin, diphtheria toxin, or truncated versions thereof. Also, included would be genes that encode prodrugs, cytokines, or chemokines. Such oncolytic virus may utilize promoters from genes that are highly expressed in the targeted tumor such as the epidermal growth factor receptor promoter (EGFr) or the basic fibroblast growth factor (bFGF) promoter, or other tumor associated promoters or enhancer elements.

One such oncolytic virus for use in the present invention is oncolytic herpes simplex virus (oHSV). The oHSV will comprise one or more exogenous nucleic acids encoding for one or more of the polypeptides described herein. Methods of generating an oHSV comprising such an exogenous nucleic acid are known in the art. The specific position of insertion of the nucleic acid into the oHSV genome can be determined by the skilled practitioner.

Oncolytic herpes simplex viruses (oHSV) are known in the art and include type 1 herpes simplex viruses and type 2 herpes simplex viruses. In one embodiment, the oHSV used in the methods, compositions, and kits of the invention is replication-selective or replication-competent such as one of the examples described herein. In one embodiment, the oHSV is replication-incompetent.

Herpes simplex 1 type viruses are among the preferred viruses, for example the variant of HSV-1 viruses that do not express functional ICP34.5 and thus exhibit significantly less neurotoxicity than their wild type counterparts. Such variants include without limitation oHSV-R3616. Other exemplary HSV-1 viruses include 1716, R3616, and R4009. Other replication selective HSV-1 virus strains that can be used include, e.g., R47Δ (wherein genes encoding proteins ICP34.5 and ICP47 are deleted), G207 (genes encoding ICP34.5 and ribonucleotide reductase are deleted), NV1020 (genes encoding UL24, UL56 and the internal repeat are deleted), NV1023 (genes encoding UL24, UL56, ICP47 and the internal repeat are deleted), 3616-UB (genes encoding ICP34.5 and uracil DNA glycosylase are deleted), G92Δ (in which the albumin promoter drives transcription of the essential ICP4 gene), hrR3 (the gene encoding ribonucleotide reductase is deleted), and R7041 (Us3 gene is deleted) and HSV strains that do not express functional ICP34.5.

oHSV for use in the methods and compositions described herein is not limited to one of the HSV-1 mutant strains described herein. Any replication-selective strain of a herpes simplex virus may be used. In addition to the HSV-1 mutant strains described herein, other HSV-1 mutant strains that are replication selective have been described in the art. Furthermore, HSV-2, mutant strains such as, by way of example, HSV-2 strains 2701 (RL gene deleted), Delta RR (ICP10PK gene is deleted), and FusOn-H2 (ICP10PK gene deleted) can also be used in the methods and compositions described herein.

Non-laboratory strains of HSV can also be isolated and adapted for use in the invention. Furthermore, HSV-2 mutant strains such as, by way of example, HSV-2 strains HSV-2701, HSV-2616, and HSV-2604 may be used in the methods of the invention.

In one embodiment, the recombinant oncolytic virus is a recombinant oHSV-1 comprising a fragment of exogenous polynucleotides encoding suppressor of cytokine signaling 4 (SOCS4) or a functional fragment thereof, wherein the fragment of exogenous polynucleotides is located between U_{L}3 and U_{L}4 genes of oHSV-1. In one embodiment, the recombinant oHSV-1 is an F strain (oHSV-1(F)).

Viral Vectors

In one embodiment of the invention, a recombinant virus is a recombinant viral vector carrying a SOCS4 encoding polynucleotide. A viral vector may also be called a vector, vector virion or vector particle. In another embodiment, the viral vector is derived from a retrovirus, adenovirus, adeno-associated virus, herpes simplex virus, vaccinia virus or baculovirus. The term “viral vector” as used in the present invention refers to a vector derived from a virus for delivering a polynucleotide (e.g., encoding SOCS4 or its variants) to a normal cell and express the polynucleotide in that cell.

A retroviral vector may be derived from or may be derivable from any suitable retrovirus. A large number of different retroviruses have been identified. Examples include: murine leukemia virus (MLV), human T-cell leukemia virus (HTLV), mouse mammary tumor virus (MMTV), Rous sarcoma virus (RSV), Fujinami sarcoma virus (FuSV), Moloney murine leukemia virus (Mo MLV), FBR murine osteosarcoma virus (FBR MSV), Moloney murine sarcoma virus (Mo-MSV), Abelson murine leukemia virus (A-MLV), Avian myelocytomatosis virus-29 (MC29) and Avian erythroblastosis virus (AEV). A retrovirus may be derived from a foamy virus.

Lentiviruses are part of a larger group of retroviruses. In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV) and bovine immunodeficiency virus (BIV). A lentiviral vector, as used herein, is a vector which comprises at least one component part derivable from a lentivirus. Preferably, that component part is involved in the biological mechanisms by which the vector infects cells, expresses genes or is replicated. The lentiviral vector may be derived from either a primate lentivirus (e.g. HIV-1) or a non-primate lentivirus. Examples of non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV). In another embodiment, the lentiviral vector is derived from HIV-1, HIV-2, SIV, FIV, BIV, EIAV, CAEV or visna lentivirus.

In another embodiment of the present invention, the viral vector may be an adenovirus vector. The adenovirus is a double-stranded, linear DNA virus that does not replicate through an RNA intermediate. Adenoviruses are double-stranded DNA non-enveloped viruses that are capable of in vivo, ex vivo and in vitro transduction of a broad range of cell types of human and non-human origin. These cells include respiratory airway epithelial cells, hepatocytes, muscle cells, cardiac myocytes, synoviocytes, primary mammary epithelial cells and post-mitotically terminally differentiated cells such as neurons. Adenoviruses have been used as vectors for gene therapy and for expression of heterologous genes. The large (36 kb) genome can accommodate up to 8 kb of foreign insert DNA and is able to replicate efficiently in complementing cell lines to produce very high titers of up to 10¹² transducing units per ml. Adenovirus is thus one of the best systems to study the expression of genes in primary non-replicative cells. The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they exist as an episome (independently from the host genome) as a linear genome in the host nucleus. Adeno-associated virus (AAV) is an attractive vector system for use in the present invention as it has a high frequency of integration and it can infect non-dividing cells. This makes it useful for delivery of genes into mammalian cells. AAV has a broad host range for infectivity. Recombinant AAV vectors have been used successfully for in vitro, ex vivo and in vivo transduction of marker genes and genes involved in human diseases. Certain AAV vectors have been developed which can efficiently incorporate large payloads (up to 8-9 kb). One such vector has an AAV5 capsid and an AAV2 ITR.

Herpes simplex virus (HSV) is an enveloped double-stranded DNA virus that naturally infects neurons. It can accommodate large sections of foreign DNA, which makes it attractive as a vector system, and has been employed as a vector for gene delivery to neurons. The use of HSV in therapeutic procedures requires the strains to be attenuated so that they cannot establish a lytic cycle. In particular, if HSV vectors are used for gene therapy in humans, the polynucleotide should preferably be inserted into an essential gene. This is because if a viral vector encounters a wild-type virus, transfer of a heterologous gene to the wild-type virus could occur by recombination. However, if the recombinant virus is constructed in a way to prevent its replication, this could be accomplished by inserting the oligonucleotide into a viral gene that is essential for replication.

The viral vector of the present invention may be a vaccinia virus vector such as MVA or NYVAC. Alternatives to vaccinia vectors include avipox vectors such as fowlpox or canarypox known as ALVAC and strains derived therefrom which can infect and express recombinant proteins in human cells but are unable to replicate. It is to be appreciated that portions of the viral genome may remain intact following insertion of the recombinant gene. An implication of this is the notion that the viral vector may retain the capacity to infect a cell and subsequently express additional genes that support its replication and possibly promote lysis and death of the infected cell.

SOCS4 and Functional Fragments or Variants

The oncolytic virus or the viral vector described herein comprises a nucleic acid sequence that encodes SOCS4 or a biologically active fragment thereof, incorporated into the virus genome in expressible form. As such the virus serves as a vector for delivery of SOCS4 to the infected cells. The invention envisions the use of various forms of SOCS4, such as those described herein, including without limitation, a functional domain of SOCS4 or therapeutic SOCS4 domain or therapeutic SOCS4 variant, and also fragments, variants and derivatives of these, and fusion proteins comprising one of these SOCS4 forms such as described herein.

The term “SOCS4” as used in this specification refers to suppressor of cytokine signaling 4, a protein containing a SH2 domain and a SOCS BOX domain. The protein thus belongs to the suppressor of cytokine signaling (SOCS), also known as STAT-induced STAT inhibitor (SSI), protein family. SOCS4 is known to be responsible for inhibition of immune signalling by signal transducer and activator of transcription 3 (STAT3) inhibition, and mice lacking functional SOCS4 are hyper-susceptible to primary infection with influenza A virus, displaying dysregulated pro-inflammatory cytokine production in the lungs, delayed viral clearance and impaired trafficking of influenza-specific CD8^{±}T cells to the site of infection. A functional fragment/variant/derivative of SOCS4 refers to a polypeptide substantially homologous to a native SOCS4, but which has an amino acid sequence different from that of native SOCS4 because of one or a plurality of deletions, insertions or substitutions. An example of the SOCS4 is from Homo sapiens with GenBank Access number NC_000014.9.

Fragments, variants and derivatives of native SOCS4 proteins for use in the invention that retain a desired biological activity of SOCS4 are also envisioned for delivery by the oncolytic virus or the viral vector. In one embodiment, the biological activity of a fragment, variant or derivative of SOCS4 is essentially equivalent to the biological activity of the corresponding native SOCS4 protein. In one embodiment, the biological activity for use in determining the activity is an activity of suppressing cytokine signaling. In one embodiment, 100% of the activity is retained by the fragment, variant or derivative. In one embodiment less than 100% of the activity is retained (e.g., 95%, 90%, 85%, 80%, 75%, 70%, 65%, 60%, 55%, 50%, 45%, 40%) as compared to the full length native SOCS4.

SOCS4 variants can be obtained by one or more additions, deletions, mutations or substitutions of native SOCS4 nucleotide sequences, for example. The variant amino acid or DNA sequence preferably is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to a native SOCS4 sequence. The degree of homology or percent identity) between a native and a mutant sequence can be determined, for example, by comparing the two sequences using freely available computer programs commonly employed for this purpose on the world wide web.

Alterations of the native amino acid sequence can be accomplished by any of a number of known techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required.

SOCS4 variants can, in some embodiments, comprise conservatively substituted sequences, meaning that one or more amino acid residues of a native SOCS4 polypeptide are replaced by different residues, and that the conservatively substituted SOCS4 polypeptide retains a desired biological activity that is essentially equivalent to that of the native SOCS4 polypeptide. Examples of conservative substitutions include substitution of amino acids that do not alter the secondary and/or tertiary structure of SOCS4.

Other Nucleic Acids

The recombinant oncolytic virus or viral vector comprising SOCS4 nucleic acid may further contain additional heterologous nucleic acid sequences (e.g., in expressible form), referred to herein as a second heterologous nucleic acid sequence, a third heterologous nucleic acid sequence, etc. Alternatively, the recombinant oncolytic virus or viral vector may contain no additional heterologous nucleic acid sequences.

Any desired DNA can be inserted, including DNA that encodes selectable markers, or preferably genes coding for a therapeutic, biologically active protein, such as interferons, cytokines, chemokines, or more preferably DNA coding for a prodrug converting enzyme, including thymidine kinase, cytosine deamindase, cyp450, and others. In one embodiment, the nucleic acid encodes a protein that inhibits tumor growth (e.g., a chemotherapeutic, growth regulatory agent) or modifies an immune response. An example of a chemotherapeutic agent is mitomicin C. In one embodiment, the nucleic acid encodes a growth regulatory molecule (e.g., one that has been lost in tumorigenesis of the tumor). Examples of such molecules without limitation proteins from the caspase family such as Caspase-9 (P55211(CASP9_HUMAN); HGNC: 15111; Entrez Gene: 8422; Ensembl: ENSG000001329067; OMIM: 6022345; UniProtKB: P552113), Caspase-8 (Q14790 (CASP8_HUMAN); 9606 [NCBI]), Caspase-7 (P55210 (CASP7_HUMAN); 9606 [NCBI]), and Caspase-3 (HCGN: 1504; Ensembl:ENSG00000164305; HPRD:02799; MIM:600636; Vega:OTTHUMG00000133681), pro-apoptotic proteins such as Bax (HGNC: 9591; Entrez Gene: 5812; Ensembl: ENSG000000870887; OMIM: 6000405; UniProtKB: Q078123), Bid (HGNC: 10501; Entrez Gene: 6372; Ensembl: ENSG000000154757; OMIM: 6019975; UniProtKB: P559573), Bad (HGNC: 9361; Entrez Gene: 5722; Ensembl: ENSG000000023307; OMIM: 6031675; UniProtKB: Q92934), Bak (HGNC: 9491; Entrez Gene: 5782; Ensembl: ENSG000000301107; OMIM: 6005165; UniProtKB: Q166113), BCL2L11 (HGNC: 9941; Entrez Gene: 100182; Ensembl: ENSG000001530947; OMIM: 6038275; UniProtKB: 0435213), p53 (HGNC: 119981; Entrez Gene: 71572; Ensembl: ENSG000001415107; OMIM: 1911705; UniProtKB: P046373), PUMA (HGNC: 178681; Entrez Gene: 271132; Ensembl: ENSG000001053277; OMIM: 6058545; UniProtKB: Q96PG83; UniProtKB: Q9BXH13), Diablo/SMAC (HGNC: 215281; Entrez Gene: 566162; Ensembl: ENSG000001840477; OMIM: 6052195; UniProtKB: Q9NR283). In one embodiment, the nucleic acid encodes an immunomodulatory agent (e.g, immunostimulatory transgenes), including, without limitation, Flt-3 ligand, HMBG1, calreticulin, GITR ligand, interleukin-12, interleukin-15, interleukin-18, or CCL17.

The exogenous nucleic acids can be inserted into the oncolytic virus by the skilled practitioner. In one embodiment, the oncolytic virus is HSV and the exogenous nucleic acid is inserted into the thymidine kinase (TK) gene of the viral genome or replacing the deleted TK gene. When the oncolytic virus comprises a second exogenous nucleic acid, the nucleic acid preferably encodes an anti-oncogenic or oncolytic gene product. The gene product may be one (e.g. an antisense oligonucleotide) which inhibits growth or replication of only the cell infected by the virus, or it may be one (e.g. thymidine kinase) which exerts a significant bystander effect upon lysis of the cell infected by the virus.

Compositions

Yet another aspect of the present invention provides a pharmaceutical composition comprising a therapeutically effective amount of a recombinant virus and a pharmaceutically acceptable carrier. The pharmaceutical composition is intended for treatment of a tumor in a subject or eliminating or reducing side effects of oncolytic tumor therapy or antivirus treatment. The recombinant virus may be prepared in a suitable pharmaceutically acceptable carrier or excipient. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologies standards.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

Methods of Treatment

Another aspect of the invention relates to a method of treating a proliferative disorder in a subject. The method comprises administering a recombinant oncolytic virus comprising the SOCS4 nucleic acid sequences described herein to the subject to thereby contact cells exhibiting undesired proliferation with an effective amount of the recombinant oncolytic virus.

In one embodiment, the proliferative disorder is a tumor and the method of the invention relates to a method for inhibiting tumor progression. An effective amount of the recombinant oncolytic virus is contacted to the tumor to thereby deliver the virus to the tumor cells. The term “tumor” or “cancer” refers to the tissue mass or tissue type or cell type that is undergoing uncontrolled proliferation. As used herein, the term “tumor” refers to a malignant tissue comprising transformed cells that grow uncontrollably (i.e., is a hyperproliferative disease). Tumors include leukemias, lymphomas, myelomas, plasmacytomas, and the like; and solid tumors. Examples of solid tumors that can be treated according to the invention include but are not limited to sarcomas and carcinomas such as melanoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma.

Methods of Eliminating or Reducing Side Effects

Another aspect of the invention relates to a method for reducing or eliminating side effects of oncolytic virus therapy in a subject comprising administering to the subject a therapeutically effective amount of a recombinant oncolytic virus, wherein the recombinant oncolytic virus comprises a fragment of exogenous polynucleotides encoding suppressor of cytokine signaling 4 (SOCS4) or a functional fragment thereof and expresses SOCS4 or the functional fragment once replication in a cancer cell.

In some embodiments, the side effects are cytokine overproductions, also referred to herein as cytokine storm. In some embodiments, the cytokine is any one or more of MCP-1, IL-1β, IL-6, TNF-α and IFN-γ. In some embodiments, clinical syndromes or outcome of the cytokine overproduction is lung tissue damages. In some embodiments, lung tissue damages are shown as any one or more of the following features: cells infiltration, mild-to-moderate dilatation, hyperemia of local capillary, thickened alveolar wall, disrupted alveolar wall, hyperemia surrounding alveolar wall, and congested immune cells.

Another aspect of the invention relates to a method for reducing or eliminating side effects of treatment of microbial infection in a subject comprising administering to the subject a therapeutically effective amount of a recombinant virus comprising a fragment of exogenous polynucleotides encoding suppressor of cytokine signaling 4 (SOCS4) or a functional fragment thereof, wherein the recombinant virus expresses SOCS4 or the functional fragment once replication in a cell. In some embodiments, the microbial infection is any one of bacterial infection, viral infection or fungal infection.

In some embodiments, the side effects are cytokine overproductions, also referred to herein as cytokine storm. In some embodiments, the cytokine is any one or more of MCP-1, IL-1β, IL-6, TNF-α and IFN-γ. In some embodiments, clinical syndromes or outcome of the cytokine overproduction is lung tissue damages. In some embodiments, lung tissue damages are shown as any one or more of the following features: cells infiltration, mild-to-moderate dilatation, hyperemia of local capillary, thickened alveolar wall, disrupted alveolar wall, hyperemia surrounding alveolar wall, and congested immune cells.

### EXAMPLES

Materials and methods

Animals: Female Balb/c mice, aged 6 weeks were purchased from Experiment Animal Center of GuangDong and housed freely from microbial pathogens at animal center of Guangzhou medical university. All procedures involving mouse were approved by the institutional animal care and use committee of Guangzhou medical university.

Cells and virus strains: Vero cells were obtained from the American Type Culture Collection and were cultured in DMEM (high glucose) supplemented with 5% (vol/vol) fetal bovine serum (FBS), or 5% (vol/vol) newborn calf serum (NBCS), respectively. HSV-1 (F), the prototype HSV-1 strain used in our laboratory was propagated and titrated on Vero cells. pReveiver-M02 with Homo sapiens suppressor of cytokine signaling 4 (SOCS4) mRNA was purchased from GeneCopoeia Inc.

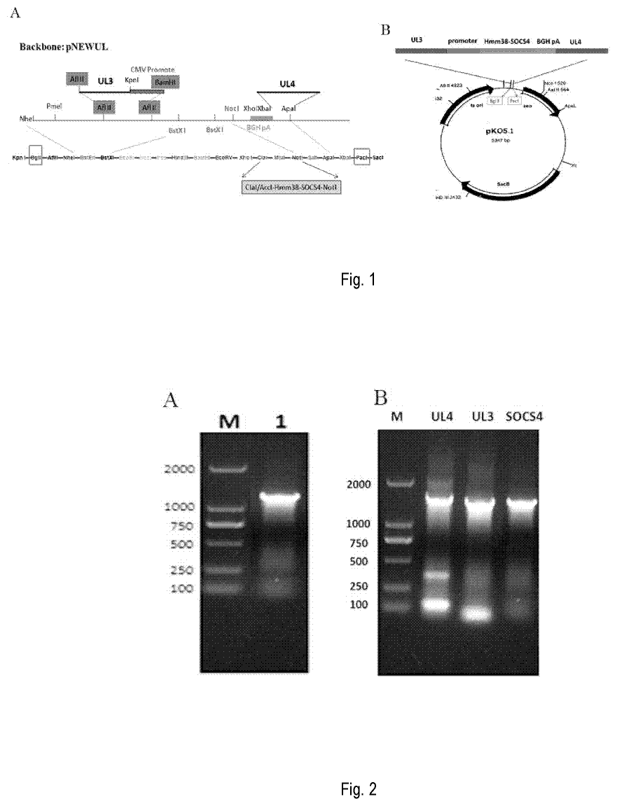

Construction of HSV recombinant virus with expression of SOCS4: Two oligonucleotide primers were designed according to Homo sapiens SOCS4 gene sequence: forward, 5′-GTCGACATGTGGTGGCGCCTGTGGTGGCTCTGCTGCTGTGGCCCATGGTGTGGGCCGCAGAAAATAATG AAAATATTAG-3′ (with an Accl site and underlined signal peptide Hmm38); reverse, 5′-GCGGCCGCCTAGCATTGCTGTTCTGGTGCATC-3′ (with an Not I site). PCR was performed in a total reaction volume of 50 μL for 30 cycles consisting of a denaturation step at 95° C. for 1 min, a primer annealing step at 58° C. for 30 secs, and a primer extension step at 72° C. for 2 mins. PCR product of SOCS4 was ligated into T-easy plasmid then transformed into XL-1 blue cells and sent for sequencing confirmation. Confirmed SOCS4 gene was cut from T-easy plasmid and ligated into carrier pNEWUL backbone site at Cla I/Acc I and Not I (FIG. 1A), then, sequence between BgIII and Pacl (including UL3, UL4 and SOCS4) was cleaved from pNEWUL and cloned into plasmid Pko5.1 at the same sites (FIG. 1B). Recombinant plasmid was transformed into BAC and cultured on LB plate with chloramphenicol and zeocin at 43° C. overnight, then, bacteria clone was picked and enriched on LB plate with chloramphenicol and sucrose at 30° C. overnight to excise plasmid Pko5.1. After been identified by SOCS4 gene PCR, positive recombinant BAC was cultured to proliferate then transformed into Vero cells by using Lipoectamine® LTX and PLUS according to instructions of manufacturer (Life Technologies Corporation). Cells were incubated till viral cytopathic effects were exhibited. Viral plaques were collected into 1 mL of milk, then went through freeze-thawed (−80° C. and 37° C.) three times to release virus. Virus were inoculated and cultured into 25 cm Vero cells (PD) and DNA was extracted to perform PCR for the final confirmation (including gene of UL3, UL4 and SOCS4). Desired virus was named HSV-SOCS4, harvested and stored at −80° C. for further use.

Infection of mice: BALB/c mice were randomly divided into 3 groups: one group was infected with HSV-1 (F), one group with HSV-SOCS4 and one group with PBS as mock infection respectively. In detail, each mouse was lightly anaesthetized then infected via the intranasal route with 106 PFU of HSV-1 (F) or HSV-SOCS4 in 30 μL of PBS or 30 μL PBS only. After infection, mice were weighed and monitored for morbidity and mortality every day for 12 days.

Serum and Bronchoalveolar lavage fluid samples collection and double sandwich ELISA: Orbital blood from each mouse was collected on day1, day3 and day7 after infection, and serum was separated then stored at −20° C. for further ELISA assay. After bleeding, mouse was sacrificed and the lung was flushed three times with 1 mL PBS through a blunted needle inserted into the trachea to collect bronchoalveolar lavage fluid (BALF). The samples were centrifuged, then supernatant was removed for ELISA assay and cells from two mice of the same group were pooled in order to obtain enough cells to perform flow cytometric analysis. For BALF and serum samples, a panel of cytokines, including MCP-1, IL-1β, IL-6, TNF-α and IFN-γ was evaluated by double sandwich ELISA. The concentration of every cytokine was determined relative to standard curve according to manufacturers instructions (Dakewe Biothech Company Limited).

Cell isolation and flow cytometric analysis: Cells harvested from BALF were treated with Tris-NH4CI to lyse erythrocytes, then washed twice and resuspended in cold RPMI 1640 medium. Spleen from sacrificed mouse was eviscerated, fully grinded and tissue was rinsed through a sterile wirescreen. Spleen cell suspensions were collected and red blood cells were lysed, then, cells were washed and re-suspended. Both BALF cells and spleen cells were counted and adjusted to 2×10⁶ cells/mL. Cells were washed and stained with surface marker: APC-CD4 (APC anti-mouse CD4 mAb, clone GK1.5), FITC-CD8a (FITC anti-mouse CD8 mAb, clone 53-6.7) and PE-CD62L (PE anti-mouse CD62LmAb, cloneMEL-14) for spleen cells and PB-CD11b (PB anti-mouse CD11b mAb, cloneM1/70) for BALF cells. All cells were stained for 10 mins, washed and resuspended in 2% formaldehyde-PBS for flow cytometric analysis on CytoFLEX flow cytometer (Beckman coulter Inc.) and CytExpert2.0 software.

Lung samples for viral titration analysis and pathological analysis: After been bled and sacrificed, lung of two mice from each group was directly removed, minced completely with cell culture media, then tissue homogenate was gathered, centrifuged and supernatant was collected for viral titration analysis. Vero cells were grown on 6-well plates at 2×10⁵/well till cells were 90% formation of the monolayer, then supernatant sample was added. After 24 hours incubation, cells were removed carefully, centrifuged and cell pellet was re-suspended in 1 mL of milk, and stored at −80° C. After 3 times of freeze-thawed process described above, released virus sample was diluted at 1:100, 1:1000 and 1:10000 with 1% NBCS respectively. 100 μL of each dilution were added in Vero cell monolayers on 6-well plates and cultured for 2 hrs, then, medium was replaced by new one (BME+1% NBCS+0.5%IgG) and cells were incubated for extra 72 hrs. After that, cells were collected and stained with 0.03% methylene blue to quantify the plaques. Pathological analysis of mouse lung (n=2, without BALF collection) from HSV-1 (F) infected, HSV-SOCS4 infected mice was performed by the department of pathology at Guangzhou medical university.

Results and Discussion

Construction of HSV-SOCS4 Recombinant Virus

We reconstructed a new HSV-1 strain with SOCS4 gene insert, which was named HSV-SOCS4 and SOCS4 protein was proved successfully expressed and we used it to infected mice intranasally to evaluate its effect on cytokine storm. We had inserted SOCS4 gene into BAC to reconstruct HSV-SOCS4 virus. Both SOCS4 gene PCR product and sequencing identification confirmed the recombinant. FIG. 2A showed the PCR product of SOCS4 (1397 bp) from pReveiver-M02, and finial PCR confirmation included fragments of UL4 (1492 bp), UL3 (1319 bp) and SOCS4 (1397 bp) as shown in FIG. 2B.

Cytokine Production in BALF and Serum after Virus Infection

Because cytokines instilled into the lungs could pass into bloodstream, provided direct communication between local and systemic response, so we collected both BALF and serum samples to detect several cytokines at the heart of the cytokine storm on day1, day3 and day7 post-infection.

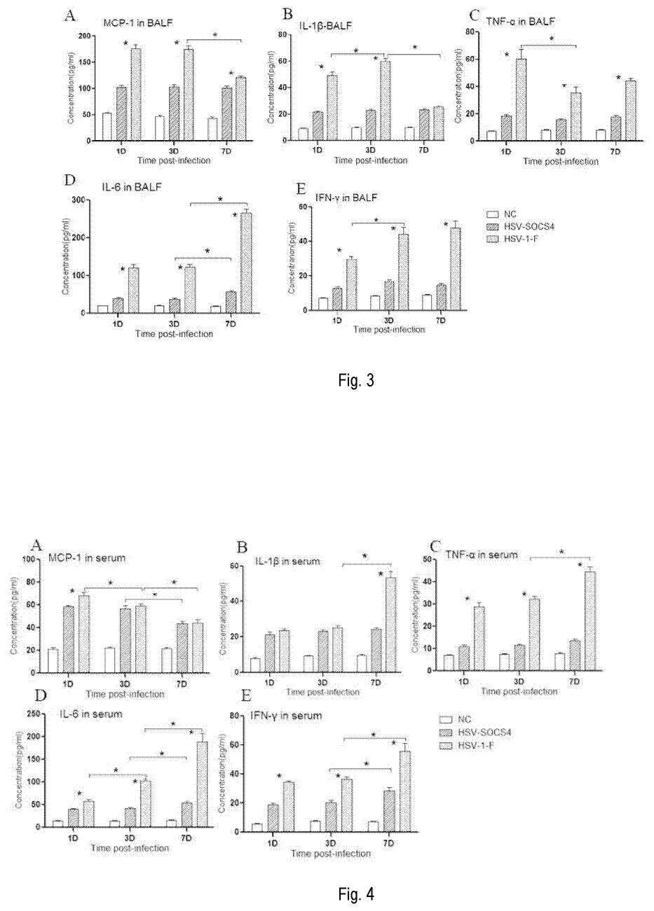

To analyzed effect of SOCS4 protein in cytokine secretion, we analysed major inflammatory cytokines, such as MCP-1, IL-1β, IL-6, TNF-α and IFN-γ profiles in both BALF and serum from mice infected with PBS or HSV-1 (F) or HSV-SOCS4 virus on day1, day3 and day7 post-infection. Mock mice did not induce appreciable amounts of cytokines in neither BALF nor serum at every time point we examined. Outcomes of cytokine production in BALF samples were shown as FIG. 3. We observed a significantly higher level of all five cytokines from HSV-1 (F) infected mice than that from HSV-SOCS4 mice on day1, day3 and day7, except IL-1β production on day7, which showed negligible difference between two groups of mice. An uptrend-downtrend curve of IL-1β production was found of HSV-1 (F) infected mice and it fleetly dropped down to about 50% on day7 (FIG. 3b). Tendency of IL-6 and IFN-γ production of HSV-1 (F) mice was increased, while the former was on day7 and the latter was on day3.

The maximum level of MCP-1 of HSV-1 (F) mice was detected on day1 and day3, then it decreased on day7. The highest TNF-α level was also observed on day1 but it declined on day3. It was interesting to find that cytokine secretion of BALF from HSV-SOCS4 infected mice was stable at analyzed time points with a slight difference, except an obvious increasing of IL-6 on day7.

In order to dissect effect of SOCS4 protein on cytokine production in system circulation, we collected mice serum for ELISA and results were shown as FIG. 4. Significant higher concentration of MCP-1 was detected of HSV-1 (F) mice than that of HSV-SOCS4 mice on day1, and its production of HSV-1 (F) mice successively decreased but it decreased only on day7 of HSV-SOCS4 mice, moreover, MCP-1 Ievel in BALF was much higher than that in serum on every day point of both groups. IL-1β values of both HSV-1 (F) and HSV-SOCS4 mice were similar on day1 and day3 but a strong upregulation on day7 was detected of HSV-1 (F) mice, therefore, great difference was found between these two group. Similar amplification pattern of TNF-α production was also observed, except the fact that values of HSV-1 (F) mice were also higher than that of HSV-SOCS4 mice on day1 and day3. Continuously increased lever of IL-6 was detected from HSV-1 (F) mice on day1, day3 and day7, but the increased lever of HSV-SOCS4 mice found only on day7, and IL-6 level of HSV-1 (F) mice were much higher than that of HSV-SOCS4 mice on every day we tested. It showed that differential escalation of IFN-γ on day1 and day 3 was slight but became evident on day7 of both HSV-1 (F) mice and HSV-SOCS4 mice. Concentration of IFN-γ in serum was higher than that in BALF on day7, which confirmed that activated T cells became the main source of IFN-γ production. To further evaluate cytokine production in serum of HSV-SOCS4 mice, we tested cytokine levels on day 12 and no notable difference was observed (data not shown). In short, cytokine production of HSV-SOCS4 infected mice in BALF was almost unanimous among time points but showed diversity in serum.

Mainly produced by DCs and macrophages, IL-1β is a key cytokine driving pro-inflammatory activity. It promoted recovery when present early in infection but is associated with a damaging inflammatory response leading to severe pathogenesis and mortality when present at late stages of infection. We found that IL-1β stayed in high lever at early stage (day 1 and day3) in BALF sample, then, degradation showed on day7; oppositely, its production in serum increased on day7 of HSV-1 (F) mice. But both BALF and serum IL-1β maintain corresponded low level during all time points of HSV-SOCS4 mice. Those data suggested that SOCS4 protein may inhibit both early IL-1β production in BALF and later production in serum.

Effects of IL-1 and IL-6 are synergistic: IL-1 is mainly expressed in the early stages of infection, followed by an increasing expression of IL-6. A production of IL-6 in BALF at site of HSV infection has been reported, and our results were unanimous: strong upregulation of IL-6 levels in BALF of HSV-1 (F) mice were evident, followed by a sustained increasing, particularly on day7, and same augment was also displayed in serum with relatively lower concentration. Restricted levels of IL-6 in both BALF and serum were apparent of HSV-SOCS4 mice with an elevation on day7. The strong later production of IL-6 was in a manner independent of the presence of virus and may relate to promote Th2 responses.

Tumor necrosis factor alpha (TNF-α), another prominent acute-response cytokine, is primarily produced by macrophages, lung epithelial cells and helper T cells, and may appear in early hours after infection. TNF-α contributes to the symptoms of severe disease after H5N1 virus infection and represents the quintessential features of cytokine storm, and it also involved in the immunopathology associated with HSV infections. TNF-α was released from both the innate immune system through virus interaction with macrophages, and NK cells at early time after infection (as the notable high level in BALF on day1 in our test), and the adaptive immune system via activation of virus specific CD4+ or CD8+ T cells, as an increased level of TNF-α in serum on day7 from HSV-1 (F) mice was observed. Variation of TNF-α level of HSV-SOCS4 infected mice were indistinguishable on every day points in both BALF and serum samples. It was reported that anti-TNF treatment can reduce the severity of weight loss and illness after H3N2 virus challenge, indicating that it may be a promising therapeutic target, and we speculated that restrained TNF production may also lessened symptoms caused by HVS infection.

Monocyte chemotactic protein-1 (MCP-1) is rapidly produced by a variety of cell types, mainly monocytes, macrophages, epithelial cells and endothelial cells following inflammatory stimuli and tissue damage. It recruits monocytes, memory T-cells, NK cells and dendritic cells to sites of tissue injury and infection, and it is typically expressed in tissue during inflammation. We observed a distinct higher level of MCP-1 in BALF at early stage from HSV-1 (F) mice, which explained that much more CD11b+ cells were found in HSV-1 (F) mice BALF sample.

IFN-γ is a potent cytokine with numerous functions, including promoting the activation of DCs and macrophages; enhancing the cytotoxicity of NK cells; and inducing antibodies production of B cells. In HSV-1 (F) infected mice, the augmented IFN-γ level shown on day3 in BALF may be produced mainly by NK cells at early stage of infection and it helped to control viral replication; and the later stage (day7) elevated level in serum from both groups of mice was because that T cells became the major source of IFN-γ, but some activities of IFN-γ had been associated with inflammation and lung injury in the later response. Moreover, prolonged and indiscrimination IFN-γ production was observed in serum sample of HSV-SOCS4 infected mice on day12 when virus could not be found in lung tissue, which confirmed that IFN-γ could be induced downstream by other cytokines or features of the immune response and SOCS4 protein may inhibited the prolonged production in serum. Pro-inflammatory cytokines are responsible for cell activation and tissue damage, additionally, the release of one cytokine may induce new cytokine production, which will in turn further cause cell and organ necrosis. The stably low level of pro-inflammatory cytokines production in both BALF and serum of HSV-SOCS4 mice were supposed to associate with the amelioration of mice lung damage compared to that of HSV-1 (F) infected mice.

Cell Analysis of BALF and Spleen after Intranasal Infection with Virus

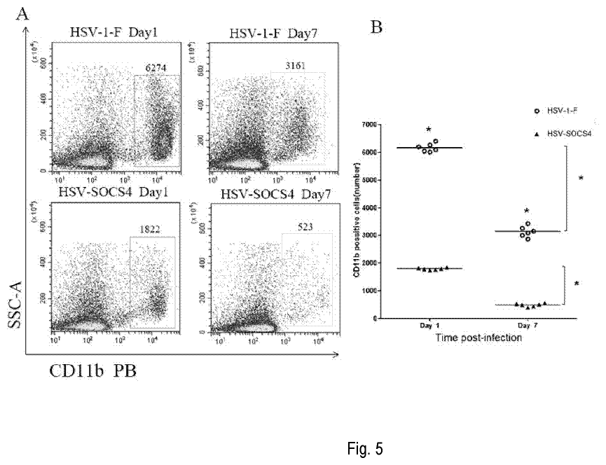

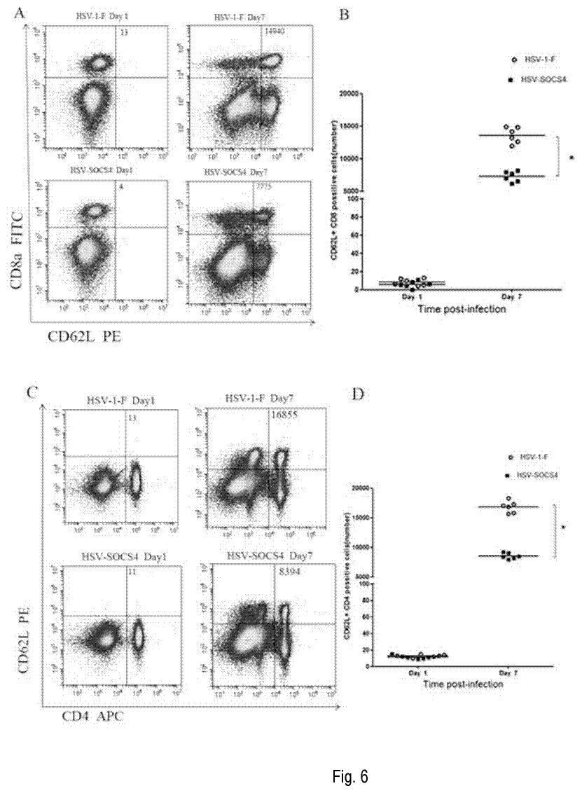

With the aim to determine whether the diverse cytokine levels in BALF and serum were related to quantity of immune cells, we collected cells from BALF and spleen from infected mice to perform flow cytometric analysis. Because the difference of cytokines production became evidently on day1 and day7, so we collected cells from day1 and day7 to make the comparison. Considering that CD11b positive cells, including macrophages, neutrophils, and NK cells constitute the main cell population present in BALF, we decided to analyze quantitative variation of CD11b+ cells between HSV-1 (F) and HSV-SOCS4 infected mice. It was shown that CD11b+ cells from HSV-1 (F) mice were predominated over that from HSV-SOCS4 mice and much more cells were stained positive on day1 than that on day7 of both groups (FIG. 5). Both CD4+ and CD8+ cells from spleen were stained and CD62L was used as an activated marker. As FIG. 6 showed, there barely had double positive cells on day1 of both groups of mice but distinctly augmented CD8+ and CD62L+ cells were detected on day7 and the distinction of positive cell number between HSV-1 (F) and HSV-SOCS4 mice was obvious. The same pattern was also observed of CD4+ and CD62L+ cells (FIG. 6).

A predominant number of CD11 b+ cells (including macrophages, monocytes, neutrophils, and NK cells) were found in HSV-1 (F) mice BALF on day1, which was the consequence of the higher level of MCP-1 in BALF. It had been reported that macrophages play an essential role in the first line of defense to HSV within the lung by rapidly secreting primary wave of pro-inflammatory cytokines, and this explained the elevated production of TNF-α, IL-1β and IL-6 in BALF of HSV-1 (F) mice on day1, because macrophages and NK cells are the main source of those cytokines at the initial response. Recruitment of macrophages into the lung and alveolar spaces is a hallmark of the initial immune response, and CD4 and CD8 T cells in spleen may reflect the activity of adaptive immune response. CD62L is generally used as activation marker of T cells and it plays a major role in directing lymphocytes to the site of infection and inflammation. No activated T cells were found in spleen on day1 from both groups but tremendous activation cells were evident on day7, and both CD62L+CD4+ T cell and CD62L+CD8+ T cell number of HSV-1 (F) mice were two-fold higher than that of HSV-SOCS4 mice, which was tightly related to the escalation level of TNF-α, IL-1β, IL-6 and IFN-γ in serum of HSV-1 (F) mice on day7. Effected Th cells and CTLs are critical for the efficient resolution of virus infection through production of cytokines and/or direct lysis of infected cells, however, these same mechanisms also contributed to pulmonary damage. Several reports indicated that acute lung injury (ALI) was directly associated with cytokine storm in the lung alveolar environment in influenza infected mice.

Virus Titers and Pathological Changes of Lung

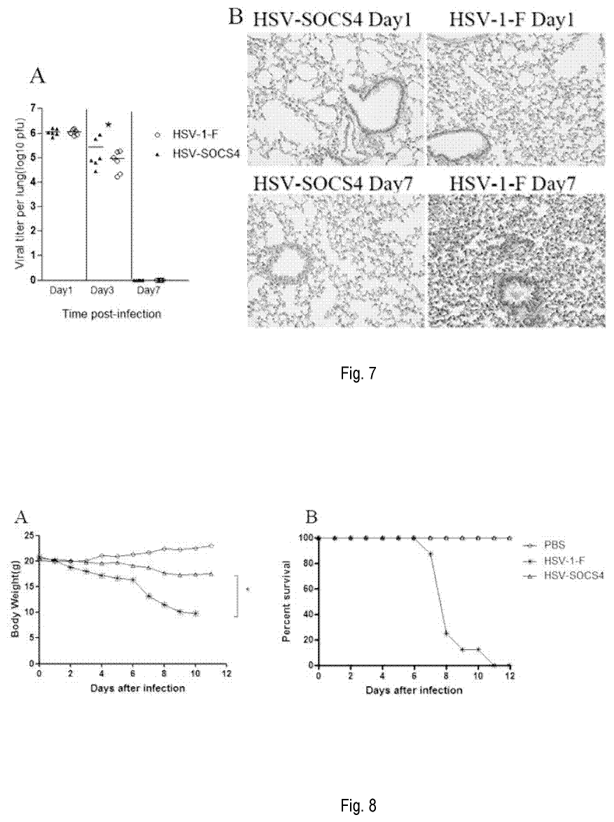

To evaluate the correlation of cytokines with virus replication/clearance, virus titer from infected mice lung was quantified. Maximum virus titer was observed on day1, it declined greatly thereafter, and no virus was detected on day7, furthermore, virus clearance displayed obvious difference between HSV-1 (F) mice and HSV-S0CS4 mice on day3 (FIG. 7A). The lungs of mice with no BALF collection were performed histopathology analysis, and a typical 200×photograph was shown as FIG. 7B. On day1, lung of HSV-SOCS4 mice barely had pathological changes; but obvious cells infiltration with slight dilatation and hyperemia of local capillary was displayed of HSV-1(F) mice lung. On day7, infiltration of some immune cells and mild-to-moderate dilatation and hyperemia of capillary were observed of HSV-SOCS4 infected mice lung but architecture of lung alveolar wall was undisrupted; a severe pathological change was appeared of HSV-1 (F) mice lung: thickened and disrupted alveolar wall with severe surrounding hyperemia, accompanied with congested immune cells.

It showed that virus titer in lung was similar between HSV-1 (F) and HSV-SOCS4 mice on day1, but it declined on day3 and obvious difference between two groups was detected. This rapid virus clearance was consistent with innate immunity mechanisms, and because of the activation of larger quantity of immune cells (as observed by flow cytometric and pathological analysis on day1), reduced virus load was detected from HSV-1 (F) mice lung. Usually, lytic infection shut off on day7, therefore, no virus was tested. After infected by HSV-1 (F), serious pathological changes of mice lungs were observed on day7, which was accordant with the increased cytokine levels and T cells of the mice.

Body Weight and Mortality of Mice after Intranasal Infection with HSV-1 (F) or HSV-SOCS4

After infected via intranasal route, all mice were monitored twice daily for a period of 12 days to determine the body weight and onset of mortality. HSV-1 (F) infected mice started to lose their body weight gradually on day2 and the loss became sharply on day7 and the final living mouse lost 50% body weight on day10. (FIG. 8A) and consistently, percent of survival rate stared to decline on day7 (FIG. 8B), then, mice died rapidly and no mouse from HSV-1 (F) group survived on day11. HSV-SOCS4 group mice lost weight slightly and generally kept 80% of weight on day12. The survival rate of HSV-SOCS4 mice maintained at 100%, which was significantly differed with that of HSV-1 (F) infected group. Mock mice showed no weight loss and no death.

As consequence of lung damage (maybe other organ lesions involved too), excessive weight loss of HSV-1 (F) mice was started at day7, and mortality rate reached at 75% on day8 and 100% on day11. On the other hand, HSV-SOCS4 mice showed only slight weight loss and no death on day12. Those results proved that controlling cytokine storm over releasing could maintain the weight, health and survival of infected mice. Our results showed that HSV with SOCS4 protein insertion inhibited cytokine over-production, immune cells excessive infiltration, alleviated lung pathological damage and reduced mortality rate of mice.

Because immunity to virus infection is multifaceted and highly complex, and the cytokines induction is a series of sprawling network with redundancy and amplified cascades, so intervention strategies should target at multiple cytokine pathways. Our attempt of recombinant an HSV-1 variants HSV-SOCS4 strain was provided a valuable tool to inhibit the cytokine storm and its disastrous consequence, which may improve oHSV clinical treatment.

It should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

1. A recombinant virus comprising a fragment of exogenous polynucleotides encoding suppressor of cytokine signaling 4 (SOCS4) or a functional fragment thereof, wherein the recombinant virus expresses SOCS4 or the functional fragment once replication in a cell.

2. The recombinant virus of claim 1, wherein the virus is an oncolytic virus or a viral vector.

3. The recombinant virus of claim 2, wherein the oncolytic virus is oncolytic Herpes Simplex Virus 1 (oHSV-1).

4. The recombinant virus of claim 3, wherein the fragment of exogenous polynucleotides is located between UL3 and UL4 genes of oHSV-1.

5. The recombinant virus of claim 2, wherein the viral vector is derived from a retrovirus, adenovirus, adeno-associated virus, herpes simplex virus, vaccinia virus or baculovirus.

6. The recombinant virus of claim 1, wherein the cell is a cancer cell.

7. The recombinant virus of claim 6, wherein the cancer cell is a cell of esophageal cancer, lung cancer, prostate cancer or bladder cancer.

8. The recombinant virus of claim 1, wherein the SOCS4 is from Homo sapiens with GenBank Access number NC_000014.9 or at least 80% identity thereto.

9. The recombinant virus of claim 1, wherein the virus comprises a further fragment of exogenous polynucleotides encoding an immunostimulatory and/or immunotherapeutic agent.

10. A pharmaceutical composition comprising a recombinant virus of claim 1, and a pharmaceutically acceptable carrier.

11. A method for treatment of cancer in a subject comprising administering to the subject a therapeutically effective amount of a recombinant oncolytic virus, wherein the recombinant oncolytic virus comprises a fragment of exogenous polynucleotides encoding suppressor of cytokine signaling 4 (SOCS4) or a functional fragment thereof and expresses SOCS4 or the functional fragment once replication in a cancer cell.

12. The method of claim 11, wherein the oncolytic virus is oncolytic Herpes Simplex Virus 1 (oHSV-1).

13. The method of claim 11, wherein the cancer cell is a cell of esophageal cancer, lung cancer, prostate cancer or bladder cancer.

14. The method of claim 11, wherein the SOCS4 is from Homo sapiens with GenBank Access number NC_000014.9 or at least 80% identity thereto.

15. The method of claim 11, wherein the recombinant oncolytic virus comprises a further fragment of exogenous polynucleotides encoding an immunostimulatory and/or immunotherapeutic agent.

16-21. (canceled)

22. A method for reducing or eliminating side effects of treatment of microbial infection in a subject comprising administering to the subject a therapeutically effective amount of a recombinant virus comprising a fragment of exogenous polynucleotides encoding suppressor of cytokine signaling 4 (SOCS4) or a functional fragment thereof, wherein the recombinant virus expresses SOCS4 or the functional fragment once replication in a cell.

23. The method of claim 22, wherein the recombinant virus is a viral vector.

24. The method of claim 22, wherein the viral vector is derived from a retrovirus, adenovirus, adeno-associated virus, herpes simplex virus, vaccinia virus or baculovirus.

25. The method of claim 22, wherein the SOCS4 is from Homo sapiens with GenBank Access number NC_000014.9 or at least 80% identity thereto.

26. The method of claim 22, wherein the microbial infection is viral, bacterial, or fungal infection.

27. The method of claim 22, wherein the side effects are cytokine overproductions.

28. The method of claim 22, wherein the side effects are lung tissue damages.
